(19) United States
(12) Patent Application Publication
(10) Pub. No.: US 2013/0319104 A1
(43) Pub. Date: Dec. 5, 2013

(54) METHODS AND SYSTEMS OF COLLECTING AND ANALYZING DRILLING FLUIDS IN CONJUNCTION WITH DRILLING OPERATIONS

(71) Applicants: Neil Patrick Schexnaider, Rayne, LA (US); Matt Hay Henderson, Inverbervie (GB)

(72) Inventors: Neil Patrick Schexnaider, Rayne, LA (US); Matt Hay Henderson, Inverbervie (GB)

(21) Appl. No.: 13/985,964

(22) PCT Filed: Feb. 17, 2011
(86) PCT No.: PCT/US11/25229

§ 371 Date: Aug. 16, 2013

(51) Int. Cl. G01N 33/24

(52) U.S. Cl. CPC G01N 33/241
USPC 73/152.42

(57) ABSTRACT

A fluid sampling system (220) includes an inline fluid extraction body (210). The inline fluid extraction body comprises an inlet (202), a first outlet (206) and a second outlet (208). A pump (214) directs a portion of a fluid flowing through the inlet into the second outlet. A flow restrictor (222) is fluidically coupled to the second outlet and regulates pressure of fluid flow through the second outlet. An extraction system (230) is fluidically coupled to the second outlet and extracts a gas sample from the fluid sample. The gas sample may then be analyzed by an analyzer.

### BACKGROUND

Drilling fluids are often circulated downhole during drilling operations. The drilling fluids perform a number of functions, including lubricating the area being drilled and removing any cuttings that are created during the drilling operations. Once the drilling fluids are returned to the surface the cuttings may be removed and the drilling fluids may be sent back downhole. As oil well drilling becomes increasingly complex, it is desirable to collect and analyze information relating to the formation.

Properties of the drilling fluid are typically monitored during drilling operations. For instance, it is often desirable to accurately measure hydrocarbon gas concentrations of the drilling fluid as it leaves the wellbore. The level of the hydrocarbon gas in the drilling fluid may affect how the well is to be drilled as well as the safety of the drilling rig and personnel involved. Moreover, the concentration of hydrocarbon gases and other components present in the drilling fluid may be indicative of the characteristics of the formation being drilled and the drilling environment.

Accordingly, the analysis of drilling fluids and the changes they undergo during drilling operations is an important factor in optimizing the drilling operations and may be important to the methods of drilling as well as the efficiency of the drilling operations. Consequently, during drilling, completion and testing of a wellbore, it is desirable to obtain analytical measurements of the fluids that are returned to the surface from the wellbore.

One proposed method for collecting and analyzing the drilling fluid involves submerging a rotor within a vessel into the drilling fluid as the drilling fluid exits the wellbore. Typically, the placement of this “gas trap” is in an open pit or header box which is exposed to atmospheric conditions. The drilling fluid is agitated as it enters into and exits out of the vessel and some of the gasses dissolved therein evaporate and escape the confines of the fluid. These vaporized gases are then collected and processed by analytical methods to determine the presence and levels of hydrocarbons and other components in the drilling fluid.

With the development of more complex systems of fluid control such as Under Balanced Drilling (“UBD”) and Managed Pressure Drilling (“MPD”) manifolds, the availability of atmospheric fluid sampling opportunities is becoming scarcer. Moreover, the drilling of high pressure and high temperature (“HPHT”) wells also underscores the desirability of having more control of the drilling fluid and pumping system needed to drill the well safely. Further, with the increasing use of UBD and MPD manifolds to control fluids and pressures during drilling operations, it is desirable to develop a parallel or inline fluid sampling method for the purpose of collecting, processing, and returning drilling fluids to the manifold system.

There are currently two common methods for collection of gaseous samples for analytical processing during drilling operations. The first method entails attaching the sample point to the primary fluid/gas separator near the atmospheric end of the manifold system. However, by the time the gas from the wellbore has entered the large volume of this separator it has typically become less significant as it has already undergone mixing with other gases and lag separation from the fluids from which it was derived. The second method entails collecting an amount of drilling fluid before the separator and processing the drilling fluid to extract any gaseous compounds that are dissolved therein. Because the sampling in the second method occurs in the main stream of fluid from the well, it will not be compromised by the mixing of any other atmospheric gases or be separated from lag by any other process. However, this method does not allow an efficient continuous sampling of the drilling fluids.

It is desirable to provide methods and systems that can continuously collect a fluid sample from a pressurized system while remaining independent from that system. The need for such methods is further increased with the increasing use of UBD and MPD manifolds.

### BRIEF DESCRIPTION OF THE DRAWINGS

These drawings illustrate certain aspects of some of the embodiments of the present invention, and should not be used to limit or define the invention.

FIG. 1 illustrates a cross sectional view of a well bore disposed in a subterranean formation in which an embodiment of the disclosed invention may be used.

FIG. 2 shows a system for collecting drilling fluids in accordance with an exemplary embodiment of the present invention.

FIG. 3 shows details of inline fluid extraction body of FIG. 2 in accordance with an exemplary embodiment of the present invention.

FIG. 4 shows a perspective view of the suction tube assembly of the inline fluid extraction body of FIG. 3 in accordance with an exemplary embodiment of the present invention.

### DETAILED DESCRIPTION

The terms “couple” or “couples,” as used herein are intended to mean either an indirect or a direct connection. Thus, if a first device couples to a second device, that connection may be through a direct connection, or through an indirect connection via other devices and connections. Similarly, the term “fluidically coupled,” as used herein is intended to mean that fluid may flow directly or indirectly, through the components that are fluidically coupled to one another. The term “uphole” as used herein means along the drillstring or the hole from the distal end towards the surface, and “downhole” as used herein means along the drillstring or the hole from the surface towards the distal end.

It will be understood that the term “oil well drilling equipment” or “oil well drilling system” is not intended to limit the use of the equipment and processes described with those terms to drilling an oil well. The terms also encompass drilling natural gas wells or hydrocarbon wells in general. Further, such wells can be used for production, monitoring, or injection in relation to the recovery of hydrocarbons or other materials from the subsurface.

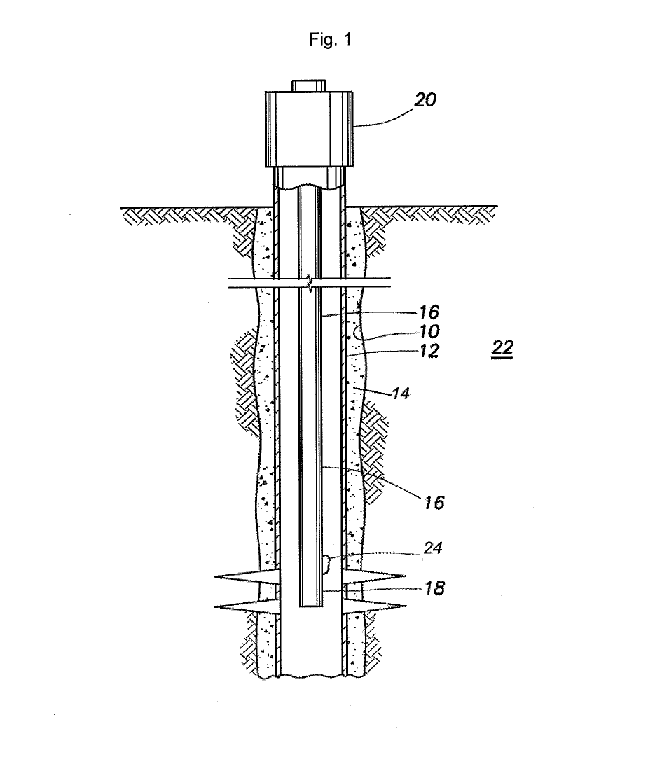

In one embodiment, the apparatus of the present disclosure may be used in a well bore disposed in a subterranean formation. Turning now to FIG. 1, a well bore 10 may be created so as to extend into a subterranean formation 22. In one embodiment, a casing 12 may be disposed within the well bore and cement 14 may be introduced between the casing 12 and the well bore 10 walls in order to hold the casing 12 in place and prevent the migration of fluids between the casing 12 and the well bore 10 walls. A tubing string 16 may be disposed within the casing 12. In an embodiment, the tubing string 16 may be jointed tubing, coiled tubing, or any other type of tubing suitable for use in a subterranean well environment. Suitable types of tubing and an appropriate choice of tubing diameter and thickness may be known to one skilled in the art, considering factors such as well depth, pressure, temperature, chemical environment, and suitability for its intended use. In an embodiment, a hydraulic workover unit 20 may be disposed at or near the top of the tubing string 16, the casing 12, or both. The hydraulic workover unit 20 may allow for tubing and other items to be introduced into the well bore 10 while a pressure exists and is maintained within the well bore 10 and tubing string 16. The existence of a pressure within the well bore may be referred to as a live well condition.

The tubing string 16 may include the drill collar 18 which is a component that provides weight on the bit for drilling and may be part of the Bottom Hole Assembly (“BHA”). Drilling related measurements may be performed downhole and information transmitted to the surface while drilling the well. Such measurements are typically referred to as Measurement While Drilling (“MWD”) operations. MWD tools may be conveyed downhole as part of the BHA. The tools used for MWD may be contained inside the drill collar 18 or built into the collar 18. One of the tools used for MWD is a collar mounted ultrasonic transducer 24 which may be mounted onto the drill collar 18.

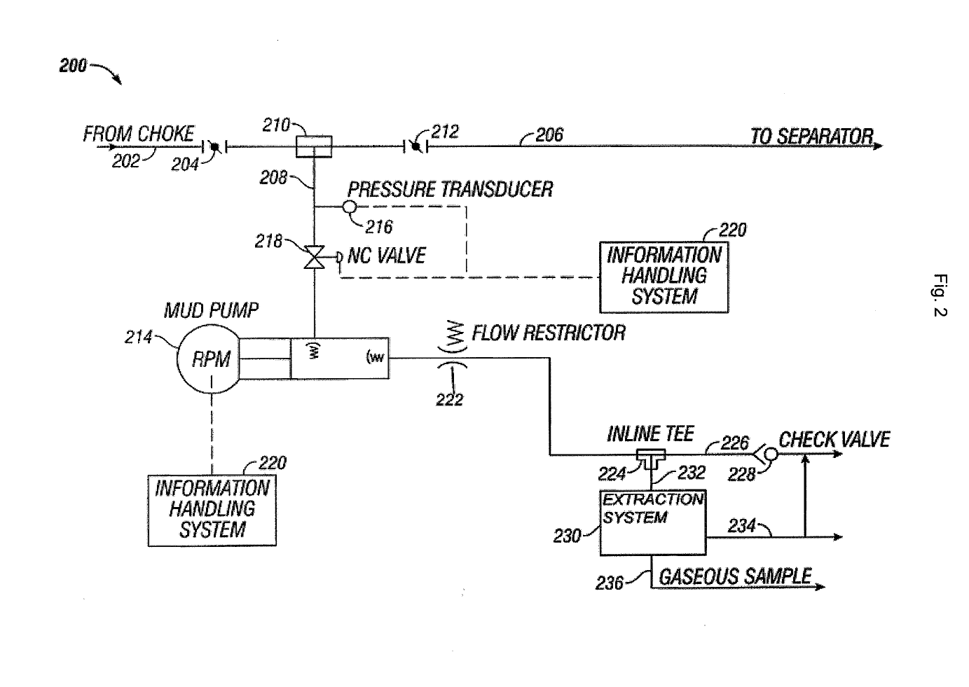

Turning now to FIG. 2, a system for collecting drilling fluids in accordance with an exemplary embodiment of the present invention (hereinafter “Sampling System”) is denoted generally with reference numeral 200. As shown in FIG. 2, the drilling fluid that is returned to the surface from the wellbore may be directed through a first inlet 202 from choke (not shown). In one exemplary embodiment, a flow meter 204 may be installed at point along the first inlet 202 to monitor the flow of drilling fluids through the first inlet 202. The first inlet 202 may split into a first outlet 206 and a second outlet 208 at an inline fluid extraction body 210. In one exemplary embodiment, the first outlet 206 may include a flow meter 212 to monitor the flow of drilling fluids through the first outlet 206. The portion of the drilling fluids that flows through the first outlet 206 may be directed to a separator (not shown). The portion of the drilling fluid that flows through the second outlet 208 is referred to herein as the drilling fluid sample. The processing of the drilling fluid in the separator is well known to those of ordinary skill in the art and will not be discussed in detail herein. In another exemplary embodiments, the flow meters 204, 212 may also be shut off valves that can regulate fluid flow through the system.

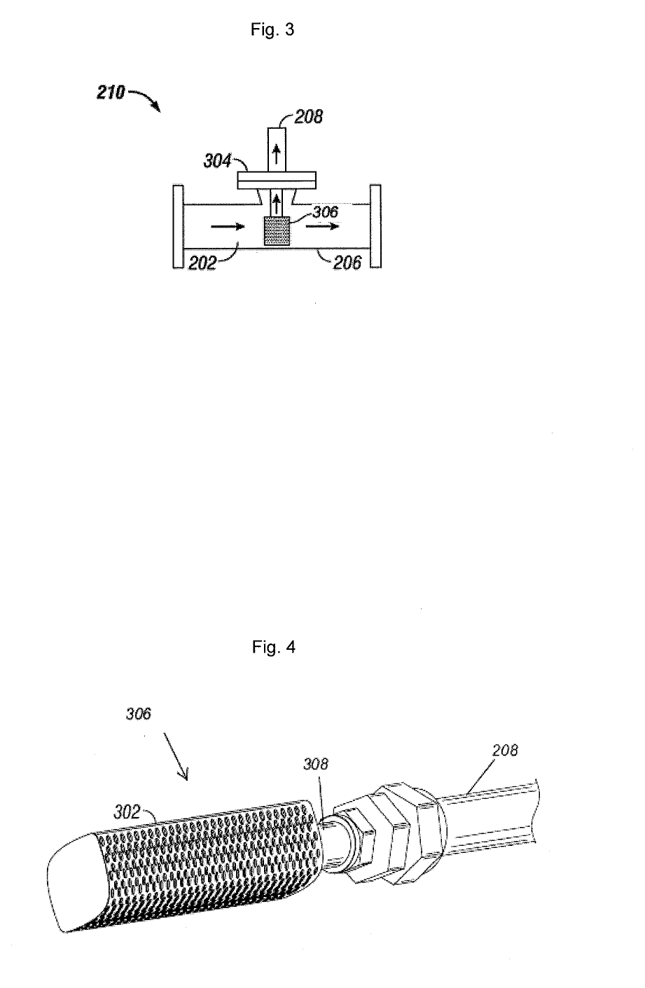

FIG. 3 depicts the details of the inline fluid extraction body 210. The inline fluid extraction body 210 is designed to maintain the pressure integrity of the manifold system while also allowing penetration and extraction of the fluids contained within. As shown in FIG. 3, the inline fluid extraction body 210 includes a suction tube assembly 306 that is fluidically coupled to second outlet 208 through a flange 304. As shown by the arrows in FIG. 3, a first portion of the drilling fluids that flow through the inlet 202 flows through the first outlet 206 and a second portion of the drilling fluids is directed through the suction tube assembly 306 to the second outlet 208.

FIG. 4 depicts the details of the suction tube assembly 306 of FIG. 3. As shown in FIG. 4, the suction tube assembly 306 may consist of a screen 302 and a pipe 308 fluidically coupled to the screen. Specifically, the fluid that passes through the screen 302 flows through the pipe 308 and exits through the second outlet 208. The screen 302 may be oriented into the direction of the drilling fluids that flow through the first inlet 202. As would be appreciated by those of ordinary skill in the art, with the benefit of this disclosure, the screen 302 mesh size may be set so as to isolate drilling fluid cuttings that are larger than a predetermined size. The portion of the drilling fluid that passes through the screen 302 is directed to the second outlet 208 through the flange 304.

In one exemplary embodiment, the screen 302 may be stainless steel and/or wing-shaped. The screen may be shaped as a wing to allow the fluid rushing by to sweep away any particulates from the holes in the screen 302. In this manner, the screen 302 may be self cleaning and may keep large solids from building up on or in front thereof. The wing shape also reduces drag on the screen 302. The drilling fluids may therefore travel smoothly around the wing shaped screen 302 and the wake caused may be reduced. Because the wing shape of the screen 302 gives rise to fewer disturbances in the fluid than a round or cylindrical object would, the intake is covered with more liquid, thus reducing the chances of atmospheric gas contamination of the sample near the rear of the screen 302. The holes in the screen 302 permit smaller cuttings and some solids to enter the second outlet 208. Because the holes in the screen 302 are the smallest diameter orifices of the entire system, any particles that pass therethrough are small enough to pass harmlessly through the system and be pumped back to the rig along with the processed drilling fluid.

Returning now to FIG. 2, the Sampling System 200 includes a pump 214. As would be appreciated by those of ordinary skill in the art, with the benefit of this disclosure, the pump 214 may be any suitable pump such as, for example, a hydraulic pump, a mud pump or a positive displacement pump. The pump 214 pulls a portion of the drilling fluid that is flowing through the first inlet 202 into the second outlet 208 through the suction tube assembly 306 of the inline fluid extraction body 210. As would be appreciated by those of ordinary skill in the art, with the benefit of this disclosure, the performance parameters of the pump 214 may be controlled by the operator. Moreover, as would be appreciated by those of ordinary skill in the art, with the benefit of this disclosure, the operations of the pump 214 may be automatically or manually controlled. The pump 214 may be used to control the flow of drilling fluids through the second outlet 208. The pump 214 facilitates the movement of the drilling fluids from a higher pressure environment in the first inlet 202 to a lower pressure environment in the second outlet 208 by forcing the fluid through a pressure regulated normally closed (“NC”) valve 218.

In one exemplary embodiment, the pump 214 may assist in making the suction tube assembly 306 self cleaning. In this embodiment, the performance parameters of the pump 214 may be used to change the force applied to pull the drilling fluid sample into the second outlet 208. For example, the force applied by the pump 214 may be increased to pull any cuttings that are clogging the screen 302 or it may be reduced so as to detach large particles that are clinging to and blocking the screen 302 in the suction tube assembly 306.

As shown in FIG. 2, in one exemplary embodiment, the Sampling System 200 may include a pressure transducer 216 to monitor the pressure of the drilling fluid sample that flows through the second outlet 208. The pressure transducer 216 may be any suitable pressure transducer, such as, for example, an electronic pressure gauge or a strain gauge. Although the systems and methods disclosed herein are not limited by the type of pressure transducer used or the pressure transducer range, in one exemplary embodiment, the pressure transducer 216 may have a range of between 0-300 psi. In one exemplary embodiment, a valve may be coupled to the second outlet 208 to regulate fluid flow through the second outlet 208. In one embodiment, the valve may be a normally closed (“NC”) valve 218. The normally closed valve 218 may be a spring loaded or an actuated valve. The NC valve 218 is closed in its “normal” condition. When actuated, the NC valve 218 may be placed in an actuated or open position allowing fluid flow therethrough. Accordingly, the NC valve 218 may regulate fluid flow through the second outlet 208. As would be appreciated by those of ordinary skill in the art, with the benefit of this disclosure, in one embodiment the NC valve 218 may be replaced with a normally open valve that closes upon actuation to prevent fluid flow through the second outlet 208.

In one embodiment, the pressure transducer 216 and/or the NC valve 218 may be communicatively coupled to one or more information handling systems 220. An information handling system 220 generally processes, compiles, stores, and/or communicates information or data for business, personal, or other purposes thereby allowing users to take advantage of the value of the information. Because technology and information handling needs and requirements vary between different users or applications, information handling systems may vary with respect to the type of information handled; the methods for handling the information; the methods for processing, storing or communicating the information; the amount of information processed, stored, or communicated; and the speed and efficiency with which the information is processed, stored, or communicated. The variations in information handling systems allow for information handling systems to be general or configured for a specific user or specific use. In addition, information handling systems may include or comprise a variety of hardware and software components that may be configured to process, store, and communicate information and may include one or more computer systems, data storage systems, and networking systems.

As would be appreciated by those of ordinary skill in the art, with the benefit of this disclosure, the pressure transducer 216 and/or the NC valve 218 may be communicatively coupled to the information handling system 220 through a wired or wireless connection. Such connections are well known to those of ordinary skill in the art and will therefore not be discussed in detail herein.

In one exemplary embodiment, the pressure transducer 216 may transmit the pressure readings to the information handling system 220. The information handling system 220 may process the pressure readings from the pressure transducer 216 in a number of ways. For instance, the information handling system 220 may log the pressure readings from the pressure transducer 216. Further, the information handling system 220 may notify the operator if the pressure in the second outlet 208 exceeds or falls below a preset threshold pressure value. This notification may be generated through an audible sound and/or a visual notification such as, for example, an electronic message or illumination of a notification light. Additionally, the information handling system 220 may be used to open and close the NC valve 218, thereby regulating fluid flow through the second outlet 208. In one exemplary embodiment, the information received from the pressure transducer 216 may be used to open or close the NC valve 218. Once the drilling fluid has reached the lower pressure environment, it may be directed into an extraction system 230 (discussed below) to separate and collect fluid and gasses contained therein to be used for analytical purposes.

The Sampling System 200 may further include a flow restrictor 222. The flow restrictor 222 may be an adjustable check valve that is operable to maintain the pressure on the upstream end of the second outlet 208. Specifically, the flow restrictor 222 may prevent the drilling fluid sample flowing through the second outlet 208 from passing through if the pressure of the drilling fluid sample is smaller than a threshold pressure value. This threshold pressure value is the pressure value that is required to open the flow restrictor 222 valve and may be set by adjusting that valve. Accordingly, the operation of the pump 214 together with the flow restrictor 222 controls the volume and pressure of the drilling fluid sample that flows through the second outlet 208.

After flowing through the flow restrictor 222, the drilling fluid sample may be directed to an Inline TEE shaped branch point 224 (“Inline TEE”). The Inline TEE 224 provides a bypass mechanism within the system that permits bypassing the extraction system 230. For instance, this bypass mechanism may allow the drilling fluid sample to be returned to the separator if/when no further analysis is desired or in the event of a system failure. Further, if the drilling fluid sample flowing through the second outlet 208 is more than the amount required for analyzing the drilling fluid, any excess drilling fluid may be directed through the first Inline TEE outlet 226. The first Inline TEE outlet 226 which may be directed to the separator (not shown) may include a check valve 228. The check valve 228 may prevent a back flow of the drilling fluid from the separator through the first Inline TEE outlet 226.

A desired amount of the drilling fluid sample may be directed to an extraction system 230 through a second Inline TEE outlet 232. The extraction system 230 may be any system suitable for extracting a gaseous sample from the drilling fluid sample. The extraction system 230 may include a fluid gas extraction system for extracting any gasses dissolved in the drilling fluid. In one exemplary embodiment, the fluid gas extraction system may be the EAGLE™ gas extraction system available from Halliburton Energy Services of Duncan, Okla. The extraction system 230 may liberate and extract dissolved gasses from drilling fluids in a controlled manner. The collected gases may then be directed to a gaseous sample outlet 236 and delivered to an one or an array of analyzers for processing. In one embodiment, the extraction system 230 may include one or more pumps for transporting the drilling fluid sample through the extraction process and returning the drilling fluid sample to the rig at the outlet 234 of the extraction system 230. The extraction system 230 may further include a heater for regulating the temperature of the drilling fluid sample and a degasser for providing a sealed method of liberating and separating dissolved gasses from the drilling fluid sample and collecting these gasses for analysis while displacing the spent liquid to be returned to the rig through the outlet 234. The extraction system 230 may further include a cooler for cooling the sample gas prior to analysis and sensors that allow the process to be continuously measured. The operations of the extraction system 230 are well known to one of ordinary skill in the art and will therefore not be discussed in detail herein.

In one exemplary embodiment, a gas analyzer (not shown) may be located in another place, building, unit or work area, separate from the extraction system 230. In this embodiment, the gas extracted from the drilling fluid by the extraction system 230 may be directed to a gas analyzer through a gaseous sample outlet 236. As would be appreciated by those of ordinary skill in the art, with the benefit of this disclosure, in another exemplary embodiment (not shown), the gas analyzer may be integrally formed with the extraction system 230. Accordingly, the extraction system 230 and the gas analyzer may be located in the same location or in different locations. Gas analyzers are well known to those of ordinary skill in the art and will therefore not be discussed in detail herein. The gas analyzers may be used to analyze the gas sample extracted from the drilling fluid. That analysis may be used to provide desirable information such as, for example, information regarding the formation being drilled or the drilling operation.

Accordingly, the methods and systems disclosed herein may be used to continuously process drilling fluids by attaching an inline collection apparatus to a drilling manifold such as a UBD or MPD manifold.

The present invention is therefore well-adapted to carry out the objects and attain the ends mentioned, as well as those that are inherent therein. While the invention has been depicted, described and is defined by references to examples of the invention, such a reference does not imply a limitation on the invention, and no such limitation is to be inferred. The invention is capable of considerable modification, alteration and equivalents in form and function, as will occur to those ordinarily skilled in the art having the benefit of this disclosure. The depicted and described examples are not exhaustive of the invention. Consequently, the invention is intended to be limited only by the spirit and scope of the appended claims, giving full cognizance to equivalents in all respects.

1. A method of analyzing a fluid comprising:
directing the fluid to an inline fluid extraction body;
wherein the inline fluid extraction body comprises a screen, a first outlet and a second outlet;
directing a first portion of the fluid through a first outlet of the inline fluid extraction body;
wherein the first portion of the fluid is directed to a separator;
directing a second portion of the fluid through a second outlet of the inline fluid extraction body;
wherein the second outlet of the inline fluid extraction body is fluidically coupled to the screen;
wherein a pump directs the second portion of the fluid through the second outlet of the inline fluid extraction body;
directing the second portion of the fluid to an Inline TEE;
directing a first portion of the second portion of the fluid to an extraction system.

2. The method of claim 1, further comprising monitoring pressure of the second portion of the fluid through the second outlet of the inline fluid extraction body using a pressure transducer.

3. The method of claim 2, wherein the pressure transducer is communicatively coupled to an information handling system.

4. The method of claim 2, further comprising notifying an operator if the pressure of the second portion of the fluid is one of less than a threshold pressure and greater than a threshold pressure.

5. The method of claim 1, further comprising regulating the pressure of the second portion of the fluid through the second outlet of the suction tube assembly using a flow restrictor.

6. The method of claim 1, further comprising directing a second portion of the second portion of the fluid to a separator.

7. The method of claim 6, wherein the second portion of the second portion of the fluid is directed to a separator through a check valve.

8. A system for continuous analysis of a drilling fluid comprising:
a first inlet;
an inline fluid extraction body fluidically coupled to the first inlet;
wherein the inline fluid extraction body comprises a suction tube assembly, a first outlet and a second outlet;
wherein the suction tube assembly comprises a screen and a pipe fluidically coupled to the screen;
wherein the first outlet of the inline fluid extraction body is directed to a separator;
wherein the second outlet of the inline fluid extraction body is directed to an extraction system;
wherein a portion of the drilling fluid that is directed through the second outlet of the inline fluid extraction body flows through the suction tube assembly; and
a pump;
wherein the pump pulls the portion of the drilling fluid that is directed through the second outlet.

9. The system of claim 8, further comprising a pressure transducer, wherein the pressure transducer monitors pressure of the portion of the drilling fluid that is directed through the second outlet of the inline fluid extraction body.

10. The system of claim 8, further comprising a flow restrictor for regulating pressure of the portion of the drilling fluid that is directed through the second outlet of the inline fluid extraction body.

11. The system of claim 8, wherein the pump is selected from a group consisting of a positive displacement pump, a mud pump and a hydraulic pump.

12. The system of claim 8, wherein the screen is self cleaning.

13. The system of claim 8, further comprising a bypass mechanism for bypassing the extraction system.

14. The system of claim 8, wherein the screen is stainless steel.

15. The system of claim 8, further comprising a valve, wherein the valve regulates fluid flow through the second outlet.

16. The system of claim 15, wherein the valve is actuated by an information handling system.

17. The system of claim 8, further comprising:
a flow restrictor fluidically coupled to the second outlet;
wherein the flow restrictor regulates pressure of fluid flow through the second outlet; and
wherein the extraction system is operable to extract a gas sample from the fluid sample.

18. The fluid sampling system of claim 17, further comprising a bypass mechanism, wherein the bypass mechanism permits bypassing the extraction system.

19. The fluid sampling system of claim 17, wherein the portion of the fluid flowing through the inlet that is directed into the second outlet flows through the screen.

20. The fluid sampling system of claim 17, further comprising a gas analyzer,
wherein the gas sample is directed to the gas analyzer, and
wherein the gas analyzer analyzes the gas sample.
